(19) United States
(12) Patent Application Publication
(10) Pub. No.: US 2013/0281553 A1
(43) Pub. Date: Oct. 24, 2013

(54) METHOD OF PRODUCING SYNTHETIC FUELS AND ORGANIC CHEMICALS FROM ATMOSPHERIC CARBON DIOXIDE

(71) Applicant: Los Alamos National Security LLC, (US)

(72) Inventors: William Louis Kubic, Los Alamos, NM (US); F. Jeffrey Martin, Los Alamos, NM (US)

(73) Assignee: Los Alamos National Security LLC, Los Alamos, NM (US)

(21) Appl. No.: 13/777,878

(22) Filed: Feb. 26, 2013

Related U.S. Application Data
(63) Continuation of application No. 12/682,442, filed on Apr. 9, 2010, now abandoned.
(60) Provisional application No. 60/979,111, filed on Oct. 11, 2007.

(51) Int. Cl. C10G 2/00; G21D 9/00; C07C 273/10

(52) U.S. Cl. CPC C10G 2/50; C07C 273/10; G21D 9/00
USPC 518/702; 585/329; 585/310; 208/46; 208/177; 564/69; 376/323

(57) ABSTRACT

The present invention is directed to providing a method of producing synthetic fuels and organic chemicals from atmospheric carbon dioxide. Carbon dioxide gas is extracted from the atmosphere, hydrogen gas is obtained by splitting water, a mixture of the carbon dioxide gas and the hydrogen gas (synthesis gas) is generated, and the synthesis gas is converted into synthetic fuels and/or organic products. The present invention is also directed to utilizing a nuclear power reactor to provide power for the method of the present invention.

### STATEMENT REGARDING FEDERAL RIGHTS

This invention was made with government support under Contract No. DE-AC52-06NA25396 awarded by the U.S. Department of Energy. The government has certain rights in the invention.

### BACKGROUND OF THE INVENTION

The United States is dependent on petroleum and natural gas for about 63% of its energy. The U.S. has less than 2% of the world's petroleum reserves and is drawing down its own reserves at a disproportionately high rate. That is, the U.S. obtains about 40% of its petroleum from domestic sources. It is likely that the peaking of oil production and increasing international demand will drive prices very high. The impact of high prices could have a profound effect on the energy sector and transportation sector. The production of plastics, petrochemicals, and other chemicals will likely also be affected. The transportation sector accounts for around 65% of the U.S. petroleum consumption and the largest components of the transportation sector are gasoline and jet fuel. The U.S. also depends on petroleum for petrochemicals, feedstocks, lubricants, solvents, and a variety of other uses. On average, about 20% of an oil barrel serves as the source of critical raw materials for the world's consumer goods. Shifting from a reliance on petroleum to electrical power cannot make up for the loss of fuel and raw materials that will result from declining petroleum availability. Therefore, it would be beneficial to have a process that provides readily available sources of hydrocarbons needed to produce liquid fuels, petrochemicals, and related goods while overcoming the significant disadvantages of existing energy technology such as carbon dioxide emissions, large scale process waste, and significant environmental and health impacts.

U.S. economic security depends on a stable supply of transportation fuel and chemicals; however, around 78% of world petroleum reserves are found in politically unstable regions. Increasing world wide competition for dwindling petroleum resources in unstable regions could compromise U.S. energy security. Therefore, it is imperative that the U.S. develop a stable and dependable energy alternative to natural resources. It would therefore be beneficial to provide reliable sources of hydrocarbons for fuels and chemicals thereby eliminating potential supply problems.

Alternative fuel sources, such as hydrogen or other liquid fuels, cannot replace petroleum in all of its uses. Alternative approaches currently being considered generally have an inherently limited capacity and application, significant technical risk, or are prohibitively expense. Therefore, it would be beneficial to provide a product that will replace petroleum but can yield the same or similar products as petroleum, is abundant, and relatively easy and inexpensive to manufacture.

The extraction and refining of fossil fuels also has a significant environmental impact. Mining coal is environmentally intrusive and exposes the environment to man-made hazards. Drilling for oil and its transportation exposes the environment to man-made hazards. Burning fossil fuels produces air pollution and carbon dioxide emissions which may lead to global warming. Therefore, it would be beneficial to provide a process for producing fuel that is a carbon-neutral source of energy and minimizes environmental impact.

One possible replacement for petroleum-based transportation fuels is hydrogen gas. However, there are technical obstacles that must be overcome in order to utilize hydrogen as a safe and economical alternative. Safe, long-distance transportation of hydrogen is expensive. Cost estimates for storing and transporting hydrogen vary between $0.90 and $6.50 per kilogram. Implementation of a hydrogen economy requires a massive investment in infrastructure which means massive expense. Also, hydrogen may be impractical for some applications such as large airliners, long-distance ground transportation vehicles, and large construction equipment. Therefore, it would be beneficial to provide an inexpensive compact alternative fuel without requiring transformation of the transportation infrastructure and technology.

Varieties of methods have been developed and are being developed for alternative fuels, however, there is a need for a process that produces fuels and organic chemicals that have the same or similar yields as natural resources and that are reliable, relatively cost-efficient, and have a low-impact on the environment. One known process for producing synthetic fuel is the Fischer-Tropsch process and it is incorporated herein by reference. The Fischer-Tropsch process is a catalyzed chemical reaction in which carbon monoxide or carbon dioxide and hydrogen are converted into liquid hydrocarbons of various forms. The principal purpose of this process is to produce a synthetic petroleum as a substitute, typically from coal or natural gas, for use as synthetic lubrication oil or as synthetic fuel. This is a process that may be used once carbon dioxide gas and hydrogen gas have been obtained, but it does not provide a mechanism for obtaining the gases. Other known processes for producing synthetic gasoline are the synthesis gas-to-synthetic methanol process and, the Mobil® Methanol-to-Gasoline process which are incorporated herein by reference. The synthesis gas-to-synthetic methanol process is a widely known method for producing methanol. Methanol may be used as a useful product and may be converted by the Mobil® Methanol-to-Gasoline process. The Mobil® Methanol-to-Gasoline process is a method of producing liquid hydrocarbons for use as synthetic fuel and organic chemicals from methanol. Again these processes may be used once carbon dioxide gas and hydrogen gas are obtained but they do not provide a mechanism for obtaining the gases. Several other processes are known in the art for converting hydrogen gas and carbon dioxide gas into fuels and organic chemicals, but it would be beneficial to provide a method of supplying the carbon dioxide gas and the hydrogen gas that is practical, reliable, and has a low-impact on the environment.

In addition, the following references are disclosed herein and incorporated herein by reference.

M. Steinberg and V-D Dang, Production of Synthetic Methanol from Air and Water Using Controlled Thermonuclear Reactor Power—I. Technology and Energy Requirement, Energy Conversion, 17:97-122 (1977), discusses use of thermonuclear power (fusion) for production of methanol but does not discuss carbon dioxide capture and recovery processes based on electrolytic stripping. This article also considers the possibility of using a nuclear cooling tower as an absorber but does not consider use of an alkaline solution as the coolant. This article does not discuss integration of the process with the energy source.

S. Stucki, A. Schuler, and M. Constantinescu, Coupled CO₂ Recovery from the Atmosphere and Water Electrolysis: Feasibility of a New Process for Hydrogen Storage, International Journal of Hydrogen Energy, 20:653-663 (1995), describes a process for producing methanol from atmospheric carbon dioxide and water using an electrolytic stripping process where absorbent solution is potassium hydroxide. A hollow fiber absorber is disclosed but not a cooling tower. Additionally, this article does not discuss the integration of the process with the energy source.

K. Sridhar et al., Combined H₂O/CO₂ Solid Oxide Electrolysis for Mars in Situ Resource Utilization, Journal of Propulsion and Power, 20: 892-901 (2004), discusses a process for using nuclear power to convert carbon dioxide from the Martian atmosphere into methane and other organic chemicals.

D. Mignard et al., Methanol Synthesis from Flue-Gas CO₂ and Renewable Electricity: a Feasibility Study, International Journal of Hydrogen Energy, 28:455-464 (2003), provides a detailed description of a process for using renewable energy to convert carbon dioxide obtained from flue gas into methanol. Integration of the process with the energy source is not considered in the article.

K. Lackner, P. Grimes, and H. Ziock, Carbon Dioxide Extraction from Air: Is It an Option?, 24^{th} Annual Technical Conference on Coal Utilization and Fuel Systems, Clearwater, Fla. (Mar. 8-11, 1999), proposes a cooling-tower-like device to absorb atmospheric carbon dioxide gas into a calcium hydroxide (lime) solution wherein air flows downward through the device as a result of evaporative cooling (the opposite direction of a cooling tower). How the carbon dioxide gas will be stripped from the calcium hydroxide solution is not discussed.

U.S. Pat. No. 4,776,171 issued Oct. 11, 1988 to Perry, Jr. et al. entitled Self-Contained Renewable Energy System discloses a process in which hydrogen gas is obtained by electrolysis of desalinized seawater and is used for solar- or wind-powered methanol production. This patent does not disclose the specific source of carbon dioxide, nor does it address energy sources other than solar and wind.

U.S. Pat. No. 4,883,823 issued Nov. 28, 1989 to Perry, Jr. et al. entitled Self-Contained Renewable Energy System discloses a process in which hydrogen gas is obtained by electrolysis of desalinized seawater and is used for solar- or wind-powered methanol production. This patent teaches that the source of carbon dioxide is alkali or alkali earth carbonates, but the carbonates are not produced by absorption of atmospheric carbon dioxide. This patent does not address energy sources other than solar and wind.

U.S. Pat. No. 5,246,551 issued Sep. 21, 1993 to Pletcher et al. entitled Electrochemical Methods for Production of Alkali metal Hydroxides without the Co-Production of Chlorine discloses a three compartment electrolytic cell for producing hydroxide from carbonates.

### SUMMARY OF THE INVENTION

In one of many illustrative, non-limiting aspects of the present invention, there is provided a method for producing synthetic fuels and organic chemicals including extracting carbon dioxide gas from the atmosphere, producing hydrogen gas, combining the extracted carbon dioxide gas and the produced hydrogen gas to produce a synthesis gas, and converting the synthesis gas to synthetic fuels and organic chemicals.

In another of many illustrative, non-limiting aspects of the present invention, there is provided a nuclear power reactor or other power source to provide power for the method of the present invention and to aid in collecting the carbon dioxide from the atmosphere. The method hereof includes extracting carbon dioxide gas from the atmosphere by absorbing the carbon dioxide gas using an absorbent solution, stripping the carbon dioxide gas from the absorbent solution using an electrolytic cell that produces a gas mixture, separating the carbon dioxide gas from the gas mixture, producing hydrogen gas using a process such as steam electrolysis, combining the carbon dioxide gas and the hydrogen gas to produce a synthesis gas, and converting the synthesis gas to a chemical product. The resultant chemical product includes synthetic fuels and organic chemicals and may be, but is not limited to, fuels, diesel fuel, gasoline, petrochemicals, plastics, butane, methanol, ethylene, propylene, aromatic compounds, petroleum derivatives, mixtures thereof, and derivatives thereof.

In yet another of many illustrative, non-limiting aspects of the present invention, there is provided a method for producing urea including extracting carbon dioxide gas from the atmosphere, producing hydrogen gas, producing nitrogen gas, combining the hydrogen gas and the nitrogen gas to produce ammonia synthesis gas, converting the ammonia synthesis gas into ammonia, and combining the extracted carbon dioxide gas and the ammonia to produce urea.

### AMENDMENT TO THE BRIEF DESCRIPTION OF DRAWINGS

Applicant respectfully requests the following be replaced in the specification of the application on page 7, beginning at line 4.

In the accompanying drawings that form a part of the specification and that are to be read in conjunction therewith and in which like reference numerals are used to indicate like or similar parts in the various views and diagrams:

FIG. 1 is a schematic diagram illustrating one embodiment of the method of the present invention;

FIG. 2 is schematic diagram illustrating one embodiment of the method of the present invention;

FIG. 3 is a schematic diagram illustrating one embodiment of the extracting step of the method of the present invention;

FIG. 4A and FIG. 4B combined (hereinafter “FIG. 4”) is a schematic diagram illustrating one embodiment of the extracting step of the method of the present invention;

FIG. 5A and FIG. 5B combined (hereinafter “FIG. 5”) is a schematic diagram illustrating one embodiment of the extracting step of the method of the present invention;

FIG. 6 is a simplified diagram in front plan cross-sectional view illustrating one embodiment of a cooling tower in accordance with the method of the present invention;

FIG. 7 is a front plan cross-sectional view illustrating one embodiment of the cooling tower in accordance with the method of the present invention;

FIG. 8 is a front plan cross-sectional view illustrating one embodiment of the cooling tower in accordance with the method of the present invention;

FIG. 9 is a front plan cross-sectional view illustrating one embodiment of the cooling tower in accordance with the method of the present invention;

FIG. 10 is a front plan cross-sectional view illustrating one embodiment of the cooling tower in accordance with the method of the present invention;

FIG. 11 is a schematic diagram illustrating one embodiment of the stripping step of the method of the present invention;

FIG. 12 is a graphical representation illustrating the cell voltage of a hydroxide cell of the present invention;

FIG. 13 is a front plan cross-sectional view of a hydroxide cell in accordance with the method of the present invention;

FIG. 14 is a schematic diagram illustrating one embodiment of the stripping step of the method of the present invention;

FIG. 15 is a graphical representation illustrating the cell voltage of the bicarbonate cell of the present invention compared to the hydroxide cell;

FIG. 16 is a front plan cross-sectional view illustrating one embodiment of a bicarbonate cell in accordance with the method of the present invention;

FIG. 17 is a schematic diagram illustrating one embodiment of a three compartment cell of the present invention;

FIG. 18 is a front plan cross-sectional view illustrating one embodiment of a mercury cell in accordance with the method of the present invention;

FIG. 19A and FIG. 19B combined (hereinafter “FIG. 19”) is a schematic diagram illustrating one embodiment of the hydrogen-producing step of the method of the present invention;

FIG. 20 is a schematic diagram illustrating possible pathways for producing fuels using the method of the present invention;

FIG. 21A and FIG. 22B combined (hereinafter “FIG. 21”) is a schematic diagram illustrating one embodiment of the combining step of the method of the present invention;

FIG. 22A and FIG. 22B combined (hereinafter “FIG. 22”) is a schematic diagram illustrating one embodiment of the method of the present invention;

FIG. 23A and FIG. 23B combined (hereinafter “FIG. 23”) is a schematic diagram illustrating one embodiment of the converting step of the method of the present invention;

FIG. 24 is a schematic diagram illustrating possible products that can be produced from methanol; and

FIG. 25A and FIG. 25B combined (hereinafter “FIG. 25”) is a schematic diagram illustrating one embodiment of the method of the present invention.

### DETAILED DESCRIPTION

There is provided herein a method of producing synthetic fuels and organic chemicals from atmospheric carbon dioxide. In one embodiment, carbon dioxide gas is extracted from the atmosphere, hydrogen gas is obtained by splitting water, a mixture of the carbon dioxide gas and the hydrogen gas (synthesis gas) is generated, and the synthesis gas is converted into synthetic fuels and/or organic products.

The method of the present invention is powered by a power source. Possible power sources include, but are not limited to, nuclear power, hydroelectric power, geothermal power, wind power, photovoltaic solar power, thermal solar power, and other appropriate power sources now known or hereafter developed. The use of a nuclear power plant is disclosed throughout but is just one example of how the process hereof can be powered. It will be appreciated by one skilled in the art that the power source may be any power source suitable for use in the method of the present invention.

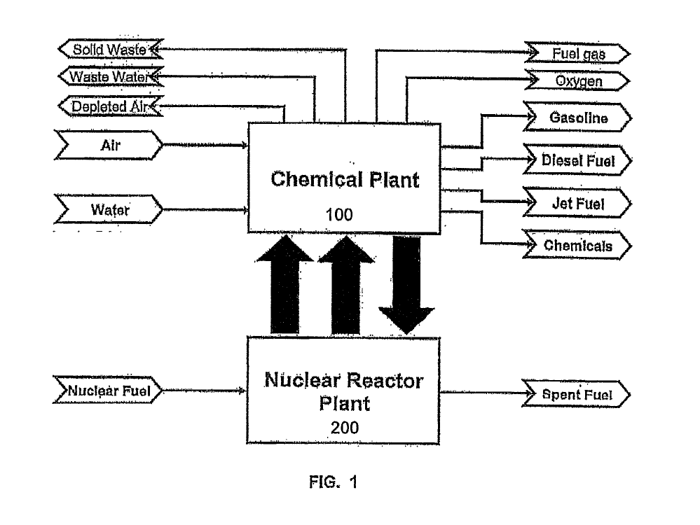

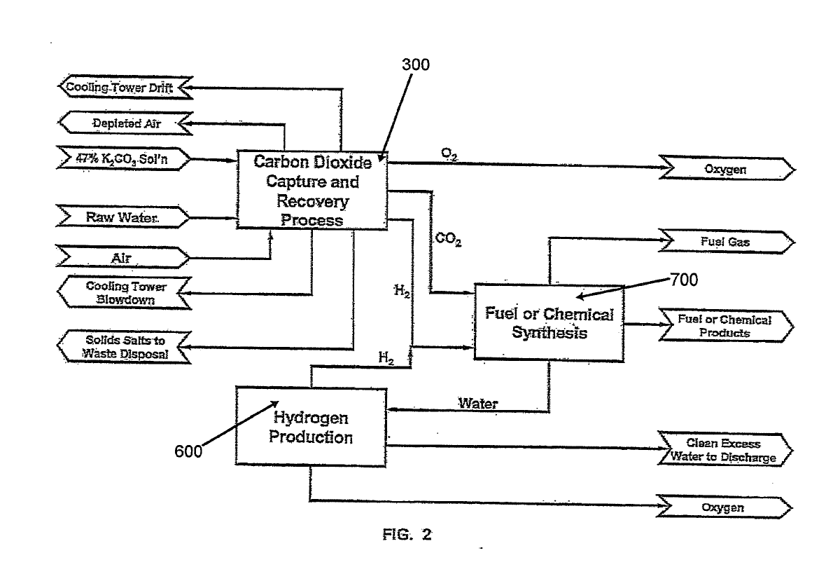

In one embodiment, illustrated by FIG. 1, a chemical plant 100 is powered by a nuclear reactor 200. Nuclear reactor 200 provides electricity and heat to chemical plant 100 thereby fueling the method of the present invention disclosed herein. There are multiple non-limiting benefits to integrating chemical plant 100 with nuclear reactor 200. One benefit of integration is having a dedicated nuclear reactor to produce electricity that is tailored to the needs of extracting the carbon dioxide gas. Another benefit is that the absorbent solution used in collecting the carbon dioxide gas may also be used to cool nuclear reactor 200. Yet another benefit is that the hydrogen produced as a byproduct of the extraction process reduces the amount of hydrogen gas needed to be produced by a separate process. Other advantages of integration include, but are not limited to: reduction of capital costs because the equipment needed has duel uses, for example, the cooling tower of nuclear reactor 200 may also be used in absorbing the carbon dioxide gas from the atmosphere; elimination of equipment, such as inverters needed to convert AC power to DC power reduces capital costs and energy consumption; collocation of nuclear reactor 200 and chemical plant 100 eliminates significant electricity transmission power losses; the process has direct access to a power system heat source; and production of hydrogen in the extraction process reduces capital costs and energy consumption needed to produce hydrogen for use later in the method.

FIG. 2 illustrates one embodiment of the method of the present invention. In this embodiment, the method for producing synthetic fuel and organic chemicals includes four steps. First, an extracting step 300 extracts carbon dioxide gas from the atmosphere (extracting step 300 may also be referred to as the capture and recovery process). Extracting step 300 also produces hydrogen as a byproduct. Next, a hydrogen producing step 600 produces hydrogen gas. Third, a combining step combines the carbon dioxide gas from the first step and the hydrogen gas from the second step to produce a synthesis gas. Finally, a converting step converts the synthesis gas to a product (both the combining step and the converting step are represented together by box 700). Possible products produced from the method of the present invention may include, but are not limited to, fuel, diesel fuel, gasoline, petrochemicals, plastics, butane, methanol, urea, ethylene, propylene, aromatic compounds, petroleum derivatives, other organic chemicals, mixtures thereof and derivatives thereof.

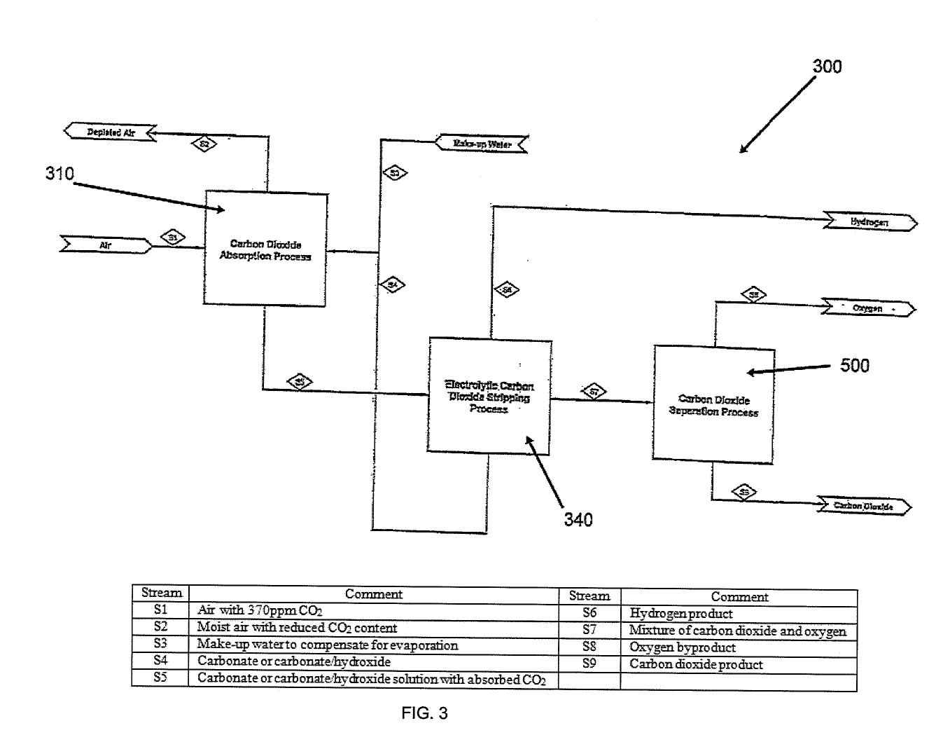

FIG. 3 illustrates one embodiment of extracting step 300. Extracting step 300, in which carbon dioxide is extracted from the atmosphere, includes three steps. First, an absorbing step 310 absorbs carbon dioxide gas in an absorbent solution. Second, a stripping step 340 is provided wherein the carbon dioxide gas is stripped from the absorbent solution and a gas mixture is produced. Third, a separating step 500 is provided wherein the carbon dioxide gas is separated from the gas mixture. Alternatively, the hydrogen may be combined with nitrogen obtained from an air separation plant to produce an ammonia synthesis gas, which is converted into ammonia. The carbon dioxide is then reacted with the ammonia to produce urea.

Extracting step 300 uses a gas-contacting device to capture the carbon dioxide gas from the atmosphere. Suitable gas-contacting devices include, but are not limited to, a natural draft cooling tower, an assisted draft cooling tower, a forced-draft cooling tower, an absorption column, an absorption column with trays, an absorption column with random packing, an absorption column with structure packing, a hollow-fiber absorber, cooling pond, spray pond, and natural alkaline lake.

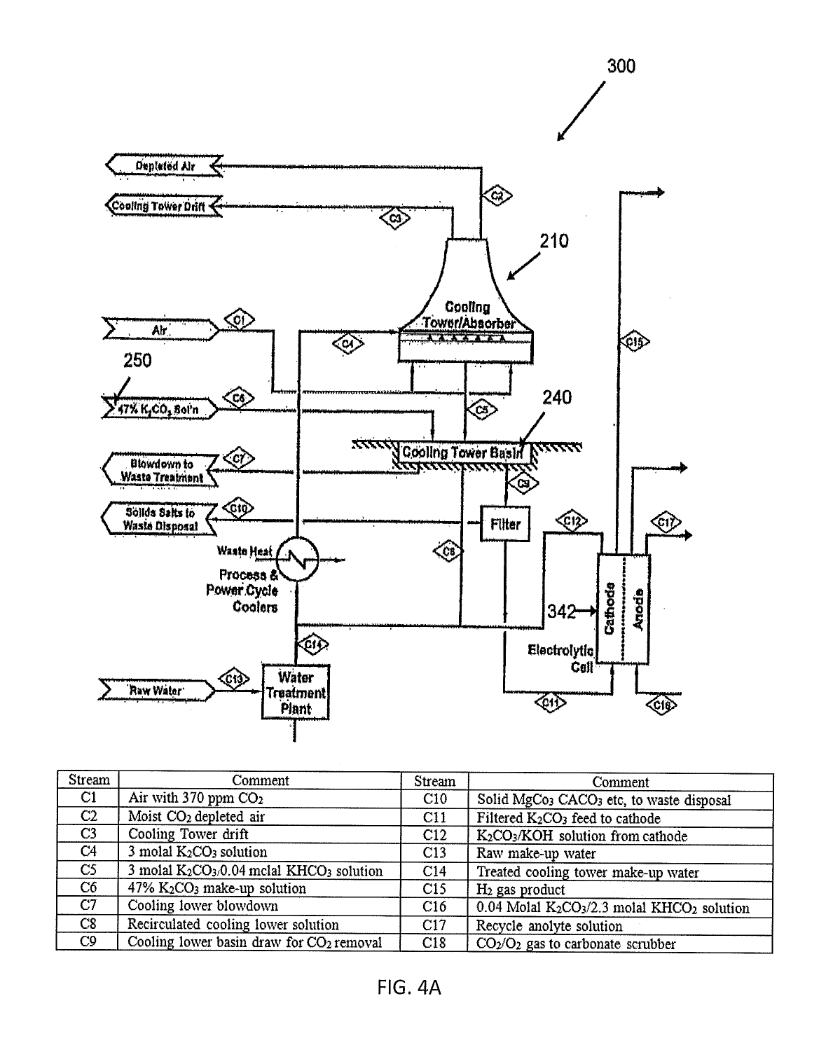

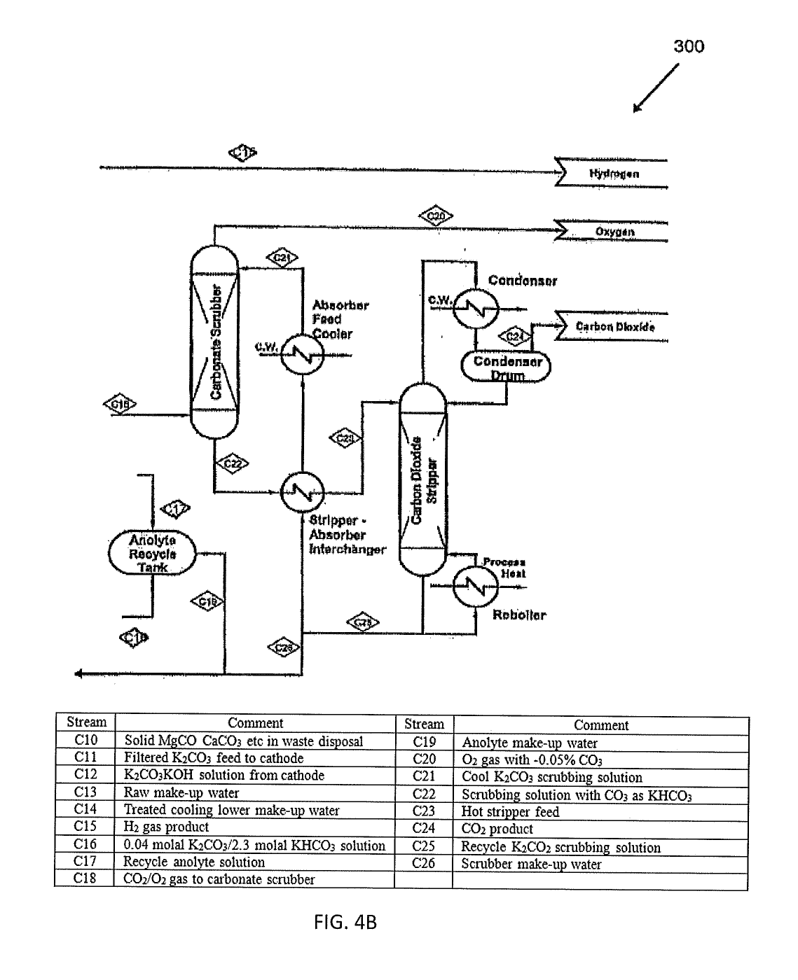

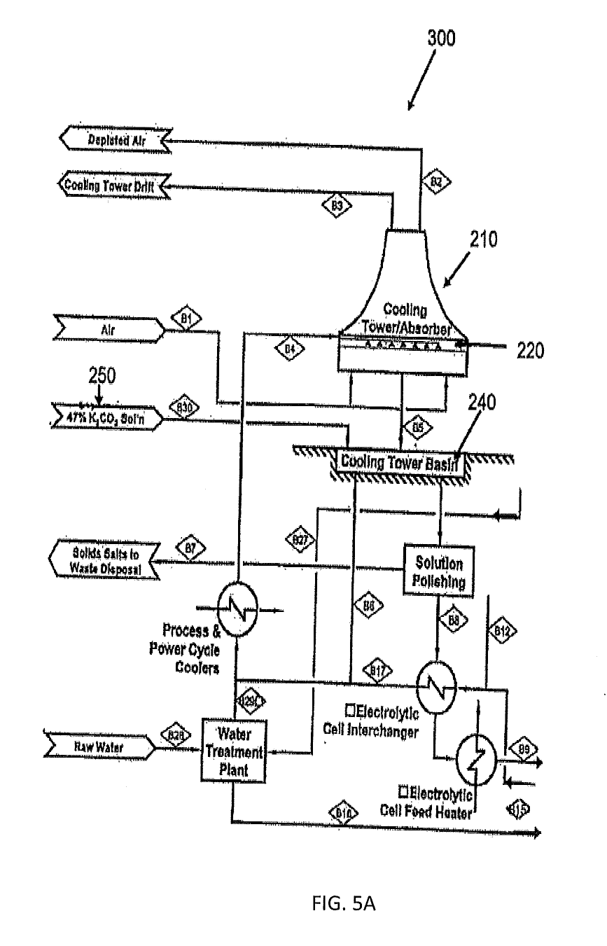

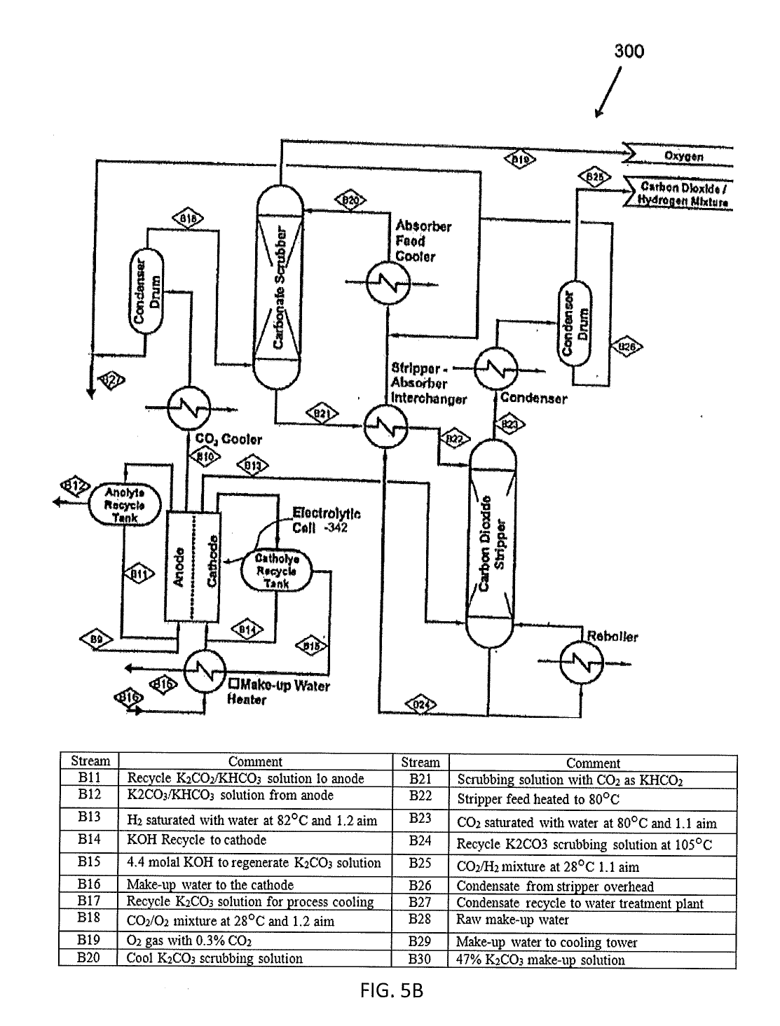

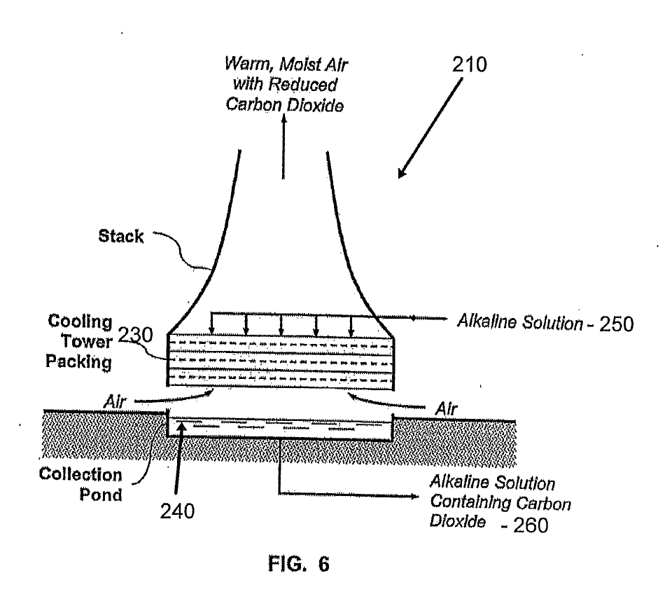

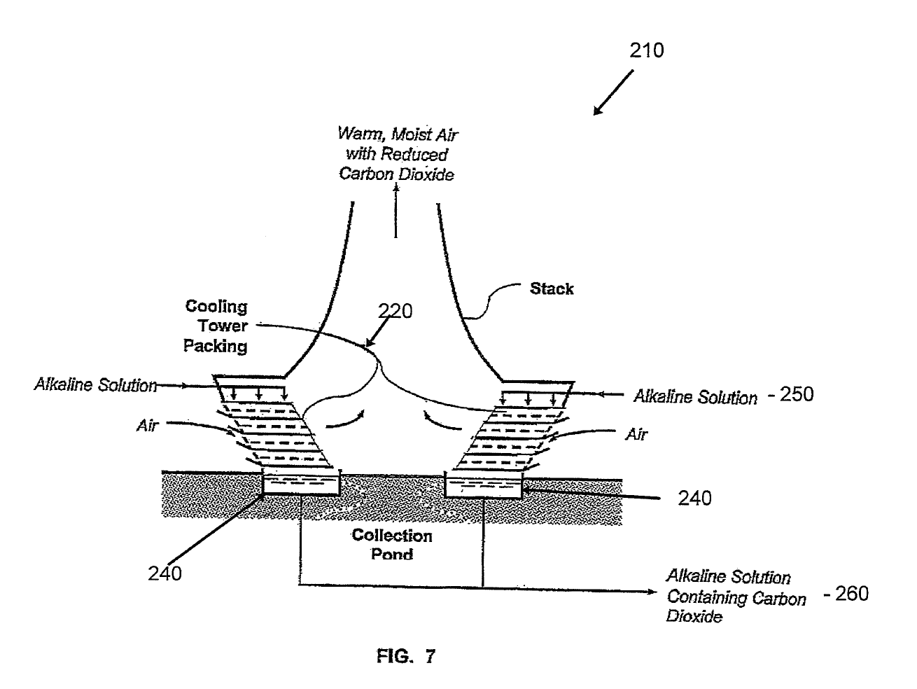

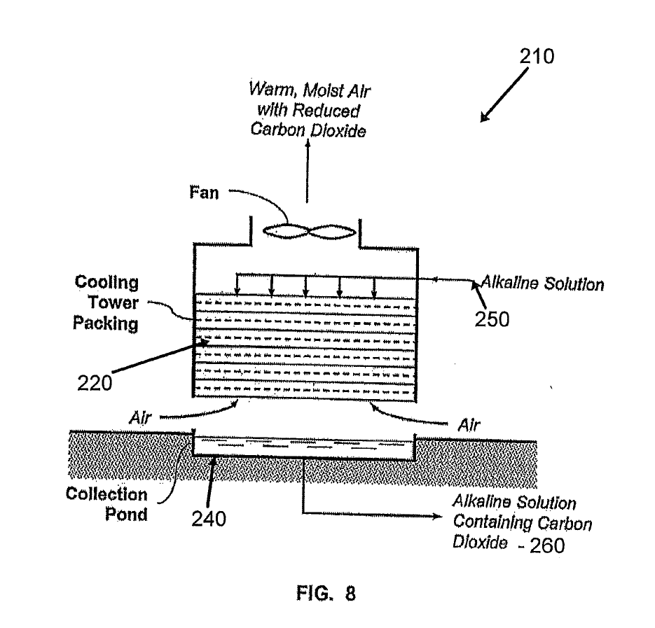

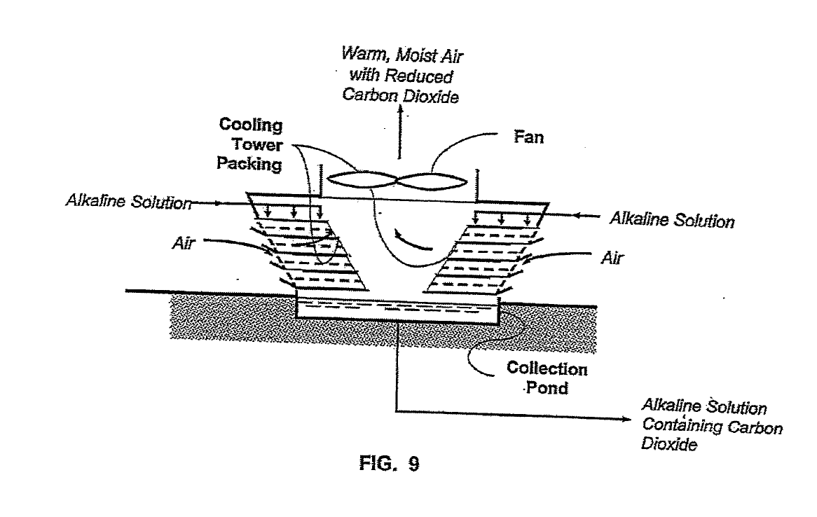

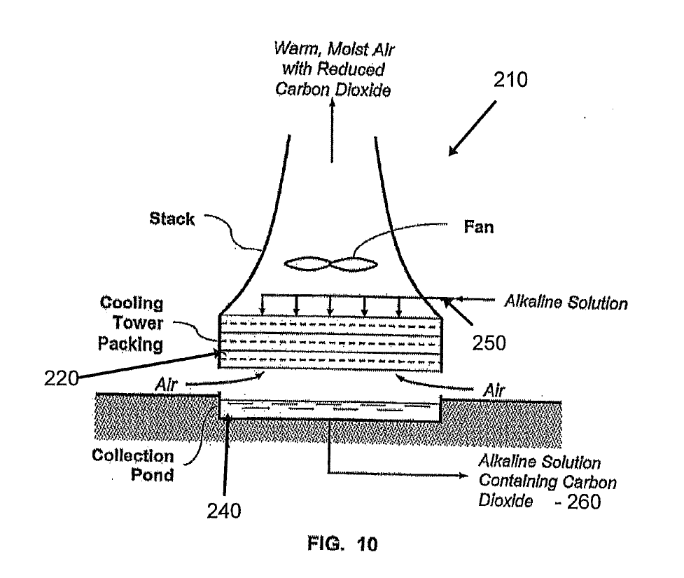

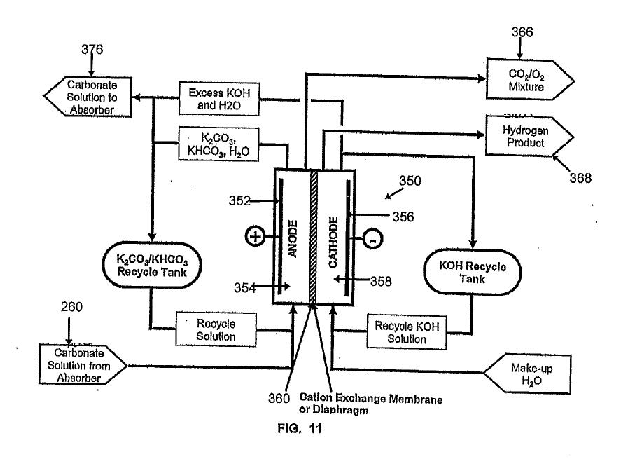

FIGS. 4 and 5 illustrate detailed and alternative embodiments of extracting step 300. As illustrated in FIGS. 4 and 5, the gas-contacting device is a cooling tower 210 of nuclear reactor 200. FIGS. 6-10 illustrate cooling tower 210 in various embodiments and configurations. Cooling tower 210 has a packing 220 which is used to circulate an absorbent solution 250 through cooling tower 210. Absorbent solution 250 is distributed over the top of packing 220. Illustrative absorbent solutions include, but are not limited to, lithium carbonate, potassium carbonate, cesium carbonate, rubidium carbonate, francium carbonate, ammonium carbonate, beryllium carbonate, magnesium carbonate, calcium carbonate, strontium carbonate, barium carbonate, potassium carbonate and potassium hydroxide mixtures, sodium carbonate and sodium hydroxide mixtures, and mixtures thereof. In an illustrative example, absorbent solution 250 preferably has a concentration from about 0.1 to 5.0 moles/liter carbonate, more preferably from about 1.0 to 5.0 moles/liter carbonate and, most preferably about 3.5 moles/liter carbonate. In one embodiment of the present invention, a potassium carbonate solution is particularly preferred for use as absorbent solution 250. One benefit of using carbonate solution as absorbent solution 250 is that concentrated carbonate solution inhibits bacteria and algae growth. Another benefit of using carbonate solution is that it may reduce scaling and corrosion in equipment. Fresh air flows through packing 220 bringing absorbent solution 250 and the air into intimate contact with each other in packing 220. The gas-liquid contact pattern can be either counter current or cross flow. Carbon dioxide gas reacts with absorbent solution 250 forming a substrate and producing a solvent 260 containing the extracted carbon dioxide. In an illustrative example, if a carbonate solution is used as absorbent solution 250, the carbon dioxide reacts with the carbonate to form a bicarbonate substrate. In another illustrative example, if a hydroxide solution is used as absorbent solution 250, carbon dioxide reacts with the hydroxide to form a carbonate substrate. In one embodiment, excess carbonate ions are present in absorbent solution 250 resulting in the very efficient capture of the carbon dioxide gas (removing over 95% of the carbon dioxide gas in the air) and reduction of carbon dioxide in a gas to 10 ppm (parts per million). Cooled solvent 260 is collected in a collection device 240 under cooling tower 210. Collection device 240 may be, but is not limited to, a basin or a collection pond (as illustrated in FIGS. 6-10). It will be appreciated by one skilled in the art that any collection device suitable for use in the present invention may be used.

As illustrated in FIGS. 4 and 5, extracting step 300 includes stripping step 340 wherein solvent 260 is processed through an electrolytic cell 342 which separates the carbon dioxide from solvent 260. Once carbon dioxide is separated from solvent 260, the remaining material may be reused in absorbing step 310 disclosed hereinabove. A suitable electrolytic cell 342 includes, but is not limited to, a hydroxide cell, a bicarbonate cell, a three compartment stripping cell, and a mercury stripping cell. In one embodiment, electrolytic cell 342 has a cathode, an anode and a diaphragm- or ion-exchange membrane separating the anode and the cathode. Water reacts at the cathode to produce hydrogen gas and hydroxide ions, represented by the following equation:

2H₂O→H₂+2OH⁻ (Reaction 1)

Hydroxide ions react at the anode to produce water and oxygen represented by the following equation:

4OH⁻→2H₂O+O₂ (Reaction 2)

The diaphragm or ion-exchange membrane separates the anode and cathode thereby preventing mixing of the solutions at the anode and cathode. Hydroxide ions accumulate on the cathode side. Consumption of hydroxide ions at the anode causes the solution at the anode to become more acidic according to the equilibrium reaction:

H₂O⇄H⁺+OH⁻. (Reaction 3)

Acidification of the solution on the anode side converts carbonate ions into bicarbonate ions and bicarbonate ions into carbon dioxide gas. These reactions are represented by the following equations:

CO₃²⁻+H⁺⇄HCO₃⁻ (Reaction 4)

HCO₃⁻+H⁺⇄CO₂+H₂O (Reaction 5)

The hydroxide ions created on the cathode side are used to regenerate absorbent solution 250. Individual electrolytic cells may be arranged in either a monopolar or bipolar fashion.

One benefit derived from using electrolytic cell 342 for stripping carbon dioxide from absorbent solution 250 is energy efficiency. An additional, but non-essential, benefit of using electrolytic cell 342 is that pure hydrogen is produced as a byproduct which can then be used in the combining step, discussed further hereinbelow. Another benefit of using electrolytic cell 342 is that oxygen gas is produced as a byproduct that may be released into the atmosphere.

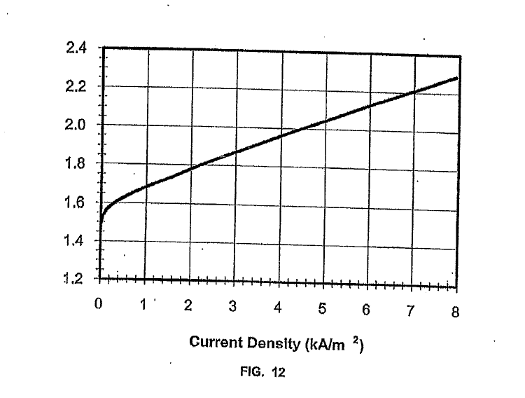

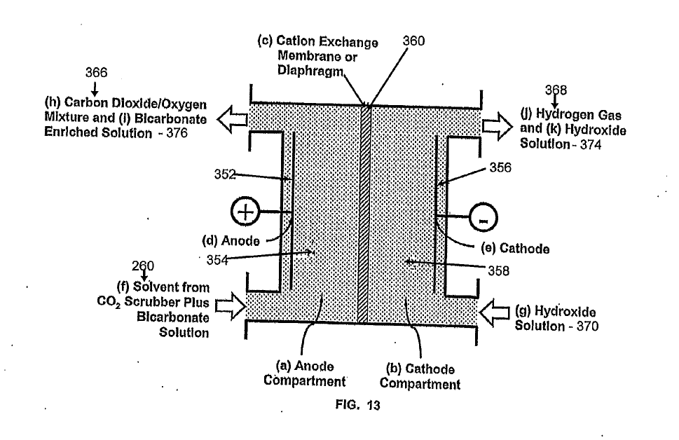

In one embodiment and as a first illustrative example, FIG. 11 illustrates stripping step 340 wherein electrolytic cell 342 is a hydroxide cell 350. Solvent 260 is fed into hydroxide cell 350 which strips solvent 260 of carbon dioxide gas produces a mixture 366 containing carbon dioxide gas and oxygen gas, and also produces hydrogen gas 368. Hydroxide cell 350 includes an anode compartment 354 containing an anode 352 and a cathode compartment 358 containing a cathode 356 separated by a membrane 360. Membrane 360 may be, but is not limited to, a diaphragm, an ion-exchange membrane, or other appropriate substance. An electrical potential is applied across hydroxide cell 350. In one embodiment, the electrical potential is from about 1.2 V to 2.5 V, more preferably from about 1.5 V to 2.2 V, and most preferably from about 1.9 V to 2.0 V. In one embodiment, hydroxide cell 350 has a current density from about 0.5 kA/m² to 10 kA/m², more preferably from about 2 kA/m² to 6 kA/m², and most preferably from about 3 kA/m² to 4 kA/m². FIG. 12 is a graphical representation illustrating cell voltage as a function of current density for one embodiment of hydroxide cell 350. In this embodiment, hydroxide cell 350 operates at a pressure of from about ambient pressure or above ambient pressure, and more preferably at about 1.3 atm. In one embodiment, hydroxide cell 350 operates at a temperature of from about 50° C. to 100° C., and more preferably at about 80° C.

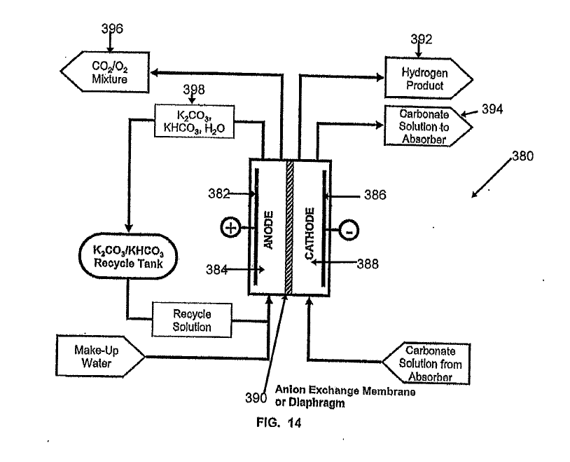

FIG. 13 is a front cross-sectional view of hydroxide cell 350. Solvent 260, containing K₂CO₃ and KHCO₃, is fed to anode compartment 354 of hydroxide cell 350. A hydroxide solution 370 is circulated through cathode compartment 358 of hydroxide cell 350. When an electrical potential is applied to hydroxide cell 350, hydroxide ions react at anode 352 to produce water and oxygen represented by Reaction 2 disclosed hereinabove. Water reacts at cathode 356 to produce hydrogen gas and hydroxide ions represented by Reaction 1. Consumption of hydroxide ions at anode 352 shifts the chemical equilibrium shown in Reaction 3, which produces hydrogen ions. Hydrogen ions react with carbonate and bicarbonate ions, illustrated by Reactions 4 and 5, and carbon dioxide is liberated (Reaction 5). Products of the reactions in anode compartment 354 include a gas mixture 366 composed of carbon dioxide gas and oxygen gas, and a solution 376 with an increased bicarbonate concentration. Gas mixture 366 is used in separating step 500 disclosed hereinbelow. Part of solution 376 is recycled to anode compartment 354 and part of solution 376 is drawn off and used to regenerate absorbent solution 250. Products of the reactions in cathode compartment 358 include hydrogen gas 372 and hydroxide solution 374. Part of hydroxide solution 374 is drawn off and used to regenerate absorbent solution 250 and part of hydroxide solution 374 is recycled to cathode compartment 358.

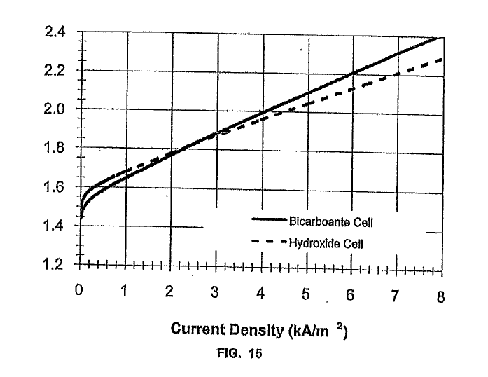

In a second embodiment and as a second illustrative example, FIG. 14 shows stripping step 340 wherein electrolytic cell 342 is a bicarbonate cell 380. Solvent 260 is fed into bicarbonate cell 380 which strips solvent 260 of carbon dioxide gas, produces a gas mixture 396 containing carbon dioxide gas and oxygen gas, and produces hydrogen gas 392. Bicarbonate cell 380 includes an anode compartment 384 containing an anode 382 and a cathode compartment 388 containing a cathode 386 separated by a membrane 390. Membrane 390 may be, but is not limited to, a diaphragm, an ion-exchange membrane, or other appropriate substance. An electrical potential is applied across bicarbonate cell 380. In one embodiment, the electrical potential is from about 1.2 V to 2.5 V, more preferably from about 1.5 V to 2.2 V, and most preferably from about 1.9 V to 2.0 V. In one embodiment, hydroxide cell 350 has a current density of from about 0.5 kA/m² to 10 kA/m², more preferably from about 2 kA/m² to 6 kA/m², and most preferably from about 3 kA/m² to 4 kA/m². FIG. 15 is a graphical representation illustrating cell voltage as a function of current density for one embodiment of bicarbonate cell 380 and comparing it with the cell voltage of one embodiment of hydroxide cell 350. In one embodiment, bicarbonate cell 380 operates at a pressure of from about ambient pressure or above ambient pressure, and more preferably at about 1.3 atm. In one embodiment, bicarbonate cell 380 operates at a temperature of from about 20° C. to 100° C., and more preferably from about 20° C. to 25° C.

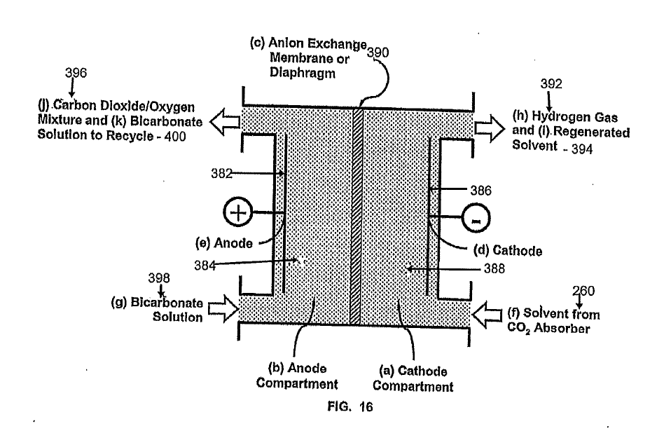

FIG. 16 is a front cross-sectional view of bicarbonate cell 380. Solvent 260, containing K₂CO₃ and KHCO₃, is fed to cathode compartment 388 of bicarbonate cell 380. A solution 398 comprising KHCO₃ mixed with water is fed to anode compartment 384 of bicarbonate cell 380. K₂CO₃ may also be added to solution 398 to control the carbon dioxide vapor pressure. When an electrical potential is applied to bicarbonate cell 380, water reacts at cathode 386 to produce hydrogen gas 392 and hydroxide ions represented by Reaction 1 disclosed hereinabove. Hydroxide ions react at anode 382 to produce water and oxygen represented by Reaction 2. Consumption of hydroxide ions at anode 382 shifts the chemical equilibrium illustrated by Reaction 3, which produces hydrogen ions. The hydrogen ions react with carbonate and bicarbonate ions, shown in Reactions 4 and 5, and carbon dioxide is liberated (Reaction 5). Products of the reactions in anode compartment 384 include a gas mixture 366 containing carbon dioxide gas and oxygen gas and KHCO₃ solution 398 is combined with make-up water and recycled back to anode compartment 384. Products of the reactions in cathode compartment 388 are hydrogen gas 392 which is removed and a carbonate solution 394. Carbonate solution 394 is recycled and used in absorbing step 310 disclosed hereinabove. Gas mixture 366 is used in separating step 500 disclosed hereinbelow.

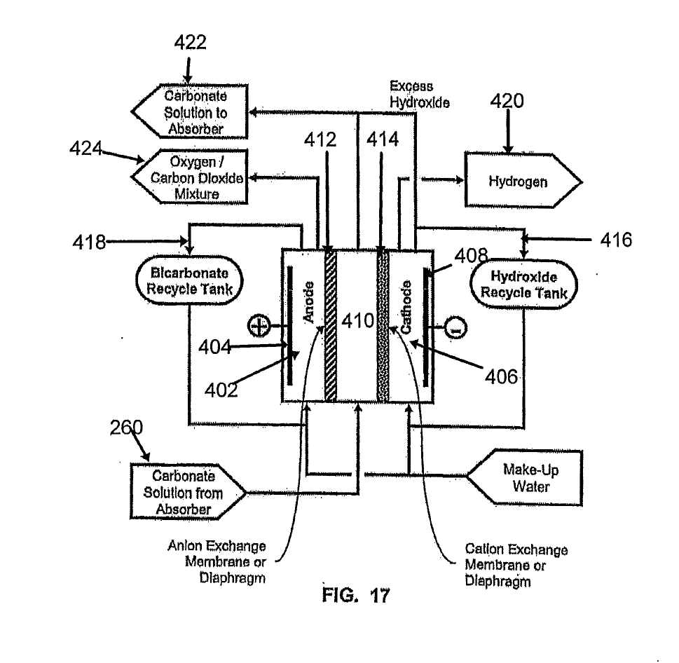

In a third embodiment and as a third illustrative example, FIG. 17 illustrates stripping step 340 wherein electrolytic cell 342 is a three compartment cell 400. Three compartment cell 400 is divided into three compartments including an anode compartment 402 having an anode 404, a cathode compartment 406 having a cathode 408, and a central compartment 410. Anode compartment 402 is separated from central compartment 410 by a first membrane 412. Cathode compartment 406 is separated from central compartment 410 by a second membrane 414. First membrane 412 may be, but is not limited to, a diaphragm, an anion-exchange membrane, or other appropriate substance. Second membrane 414 may be, but is not limited to, a diaphragm, a cation-exchange membrane, or other appropriate substance. A hydroxide solution 416 is circulated through cathode compartment 406. A bicarbonate solution 418 is circulated through anode compartment 402. Solvent 260 is fed to central compartment 410. Water reacts at cathode 408 to form hydrogen gas 420 and hydroxide ions. Cations from central compartment 410 diffuse through membrane 414 to maintain electroneutrality. Hydroxide ions react at anode 404 to form water and oxygen. The change in pH at anode 404 releases carbon dioxide from solvent 260 and a gas mixture 424 including oxygen gas and carbon dioxide gas is released from anode compartment 402. Carbonate and bicarbonate ions from central compartment 410 diffuse through membrane 412 to replenish the bicarbonate ions. The solution exiting central compartment 410 is combined with the excess hydroxide to form a carbonate solution 422 that is used to regenerate absorbent solution 250. Gas mixture 424 is used in separating step 500 disclosed hereinbelow.

In an alternative embodiment, electrolytic cell 342 is a three compartment cell (not shown) where the three compartment cell including an anion compartment (not shown) having an anion (not shown), a cation compartment (not shown) having a cation (not shown) and a central compartment (not shown). The anode compartment is separated from central compartment by a first membrane (not shown). The cathode compartment is separated from central compartment by a second membrane (not shown). The first membrane may be, but is not limited to, a PEM, a diaphragm, an anion-exchange membrane, or other appropriate substance. The second membrane may be, but is not limited to, a diaphragm, a cation-exchange membrane, or other appropriate substance. A hydroxide solution (not shown) is circulated through the cathode compartment. A bicarbonate solution (not shown) is circulated through the anode compartment. The solvent is fed to the central compartment. Oxygen forms at the anode and reacts with the hydrogen to form water and produce a current. Acidification of the solution at the anode releases a gas (not shown) from the solution. The gas produced at the anode includes carbon dioxide and water vapor. The overall energy consumption is expected to be about 40-50% less than the hydroxide cell.

In another alternative embodiment of the three compartment cell, the second membrane is an anion exchange membrane. The solvent is fed into the cathode compartment. The bicarbonate solution is circulated through the central compartment. Hydrogen from the cathode is fed to a PEM that functions as the anode. Oxygen forms at the anode and reacts with the hydrogen to form water and produce a current. Acidification of the solution at the anode releases a gas of carbon dioxide and water vapor. Overall energy consumption is expected to be about 40-50% less than the hydroxide cell.

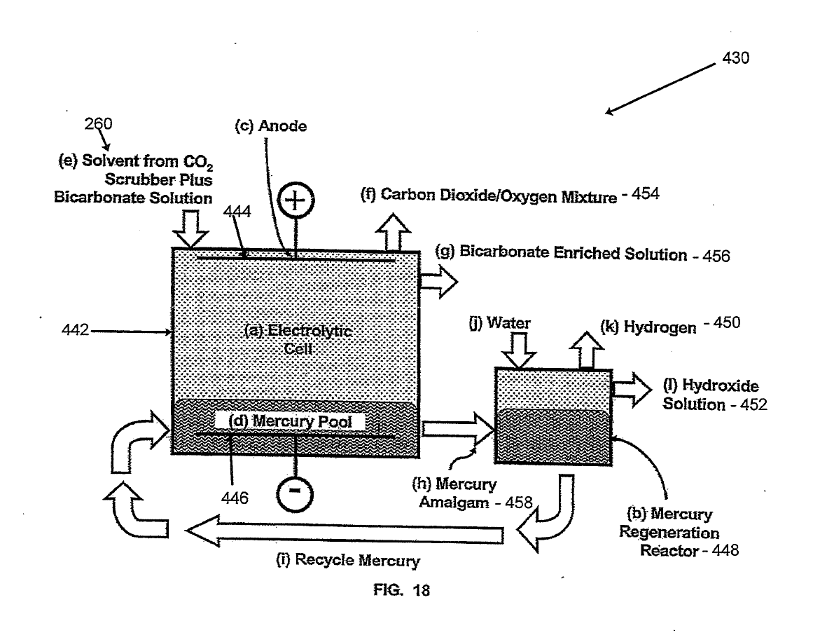

Referring to FIG. 18 illustrating a fourth embodiment, electrolytic cell 342 that is used in stripping step 340 is a mercury cell 430. Mercury cell 430 includes a compartment 442 having an anode 444 at the top of mercury cell 430 and a cathode 446 that is a pool of mercury at the bottom of compartment 442. Mercury cell 430 is connected to a mercury regeneration reactor 446. Solvent 260 is fed to compartment 442 at anode 444. In one embodiment, solvent 260 is mixed with a bicarbonate solution to improve carbon dioxide recovery. When an electrical potential is applied to mercury cell 430, hydroxide ions react at anode 444 to produce oxygen and water as represented by Reaction 2. Consumption of hydroxide ions at anode 444 reduces the pH which shifts the chemical equilibrium of Reaction 3 and produces hydrogen ions. Hydrogen ions react with carbonate and bicarbonate ions as represented by Reactions 4 and 5, and liberate carbon dioxide (Reaction 5). The products of the anode 444 reactions include a gas mixture 454 containing oxygen gas and carbon dioxide gas and an enriched bicarbonate solution 456. Gas mixture 454 is used in separating step 500 disclosed hereinbelow. Potassium, or other alkali metal, is reduced at cathode 446 to potassium metal, which forms an amalgam 458 with mercury. Mercury amalgam 458 is removed from compartment 442 and fed to mercury regeneration reactor 448. Mercury amalgam 458 is mixed with water in mercury regeneration reactor 448. The water reacts with the potassium to form potassium hydroxide solution 452, and hydrogen gas 450. It will be appreciated by one skilled in the art that any alkali metal or other appropriate element may be used in the place of potassium. The mercury is recycled to compartment 442. Hydroxide solution 452 is combined with enriched bicarbonate solution 456 to regenerate absorbent solution 250.

The third and final step of extracting step 300 is separating step 500. The gas mixture produced by electrolytic cell 342 from stripping step 340 is made up of oxygen gas and carbon dioxide gas. In one embodiment, the mixture produced contains from about 64-66% carbon dioxide on a dry basis. This mixture must be separated to obtain pure carbon dioxide. Separating step 500 may be any process suitable for separating the mixture including, but not limited to, a standard carbonate scrubber, an amine scrubber, a methanol scrubber, a pressure swing absorber, a membrane separator, or processes now known or hereafter developed that are suitable for separating carbon dioxide gas from the gas mixture. A carbonate scrubber is capable of recovering greater than 99% of the carbon dioxide gas in the gas mixture.

It will be appreciated by one skilled in the art that some processes such as the known cell for producing hydroxide from carbonates that are used for stripping step 340 may produce a pure or nearly pure carbon dioxide gas product thereby rendering separating step 500 unnecessary.

The third step in the method of the present invention is hydrogen producing step 600. Hydrogen producing step 600 may be any process that produces hydrogen gas including, but not limited to, water electrolysis, steam electrolysis, thermochemical processes, such as, for example an iodine-sulfate process, or any other suitable process now known or hereafter developed. In one embodiment, wherein hydrogen producing step 600 is water electrolysis, water electrolysis may be powered by current pressurized water reactors or boiling water reactors. Water electrolysis may be powered by pressurized water reactors or boiling water reactors which avoid a potentially large source of technical risk associated with high-temperature reactors.

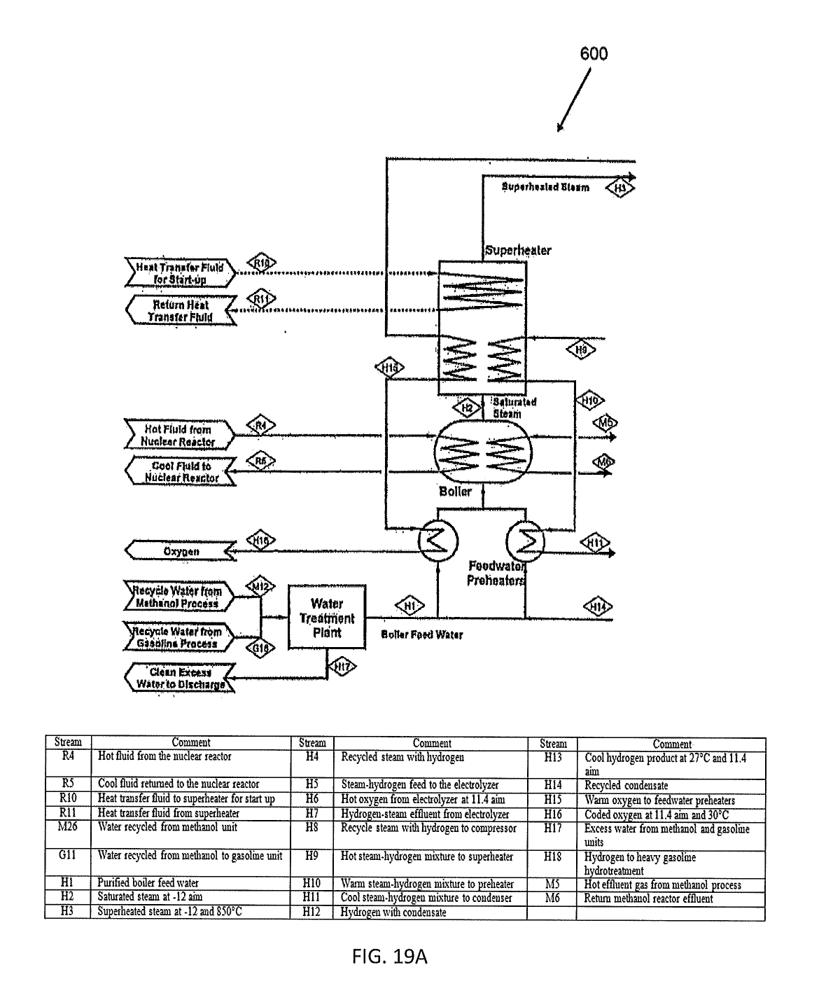

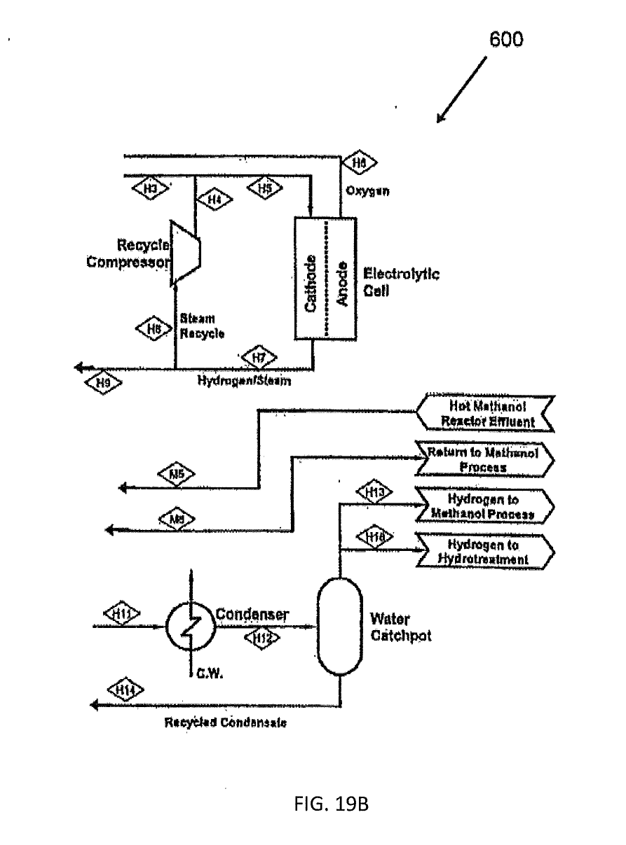

In another embodiment, illustrated by FIG. 19, hydrogen producing step 600 is steam electrolysis. Steam electrolysis utilizes a solid-oxide membrane to split steam into hydrogen and oxygen at high temperatures. Utilizing an energy integration scheme enables a stem-electrolysis cell to operate at high temperatures without requiring a high-temperature energy source. The electrolyzer heats the hydrogen and oxygen products to a temperature greater than the inlet steam. Therefore, the heat energy contained in the products can be used to superheat the steam. In effect, the electrolyzer acts as a resistance heater that produces the high temperatures needed for efficient operations. As such, it can be powered by pressurized water reactors or boiling water reactors and thereby avoiding any added technical risk of relying on a high-temperature reactor.

The combining step of the method of the present invention combines the carbon dioxide gas produced in extracting step 300, the hydrogen gas produced in extracting step 300, and the hydrogen gas produced in hydrogen producing step 600 to produce a synthesis gas.

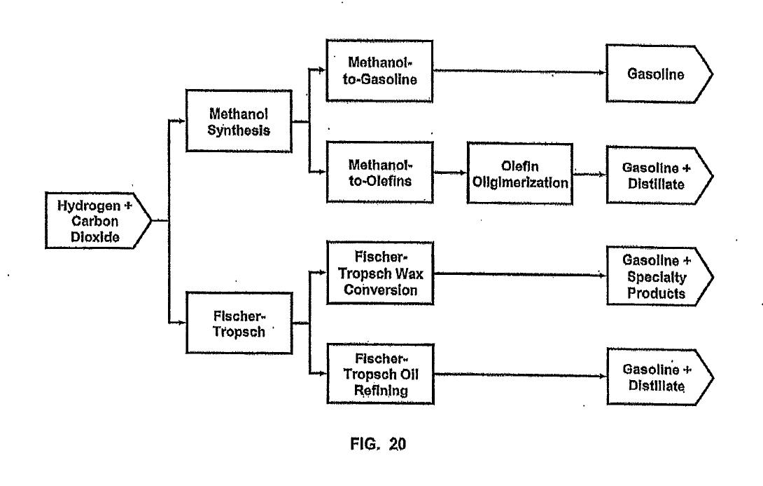

The converting step of the method of the present invention converts the synthesis gas into synthetic fuels and/or organic products. In one embodiment, the Fischer-Tropsch process is used to produce synthetic petroleum which can be refined to chemicals and fuels. In another embodiment, methanol may be produced using a methanol synthesis process and then the methanol may be further converted into fuels and chemicals. FIG. 20 illustrates possible pathways for producing different fuels and other useful products.

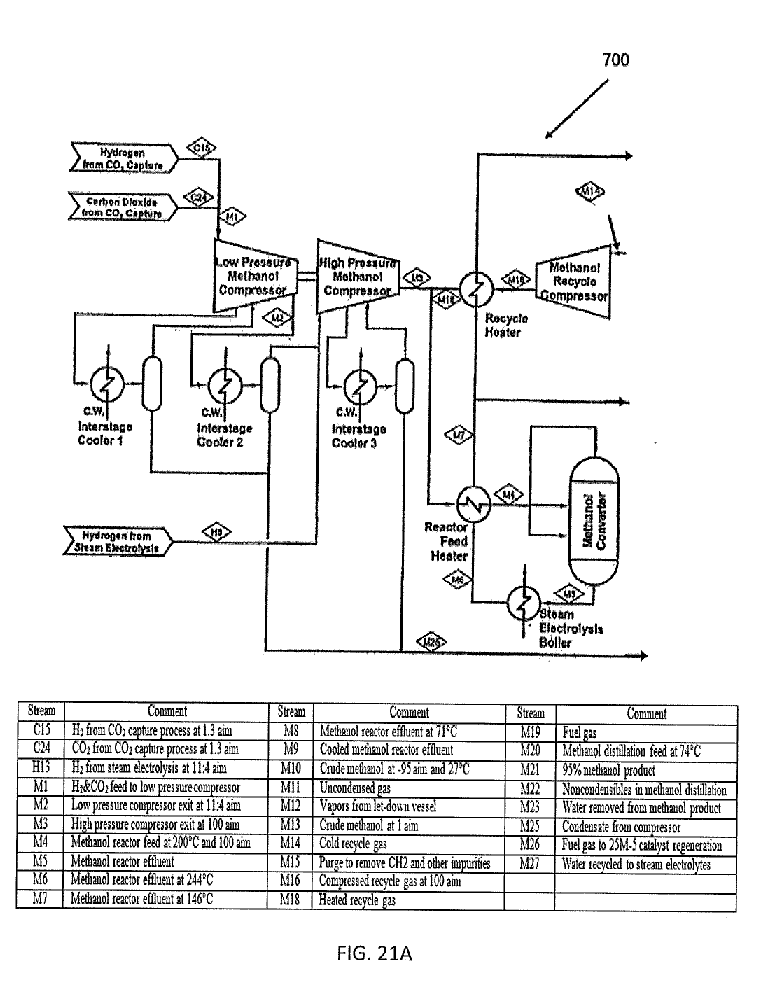

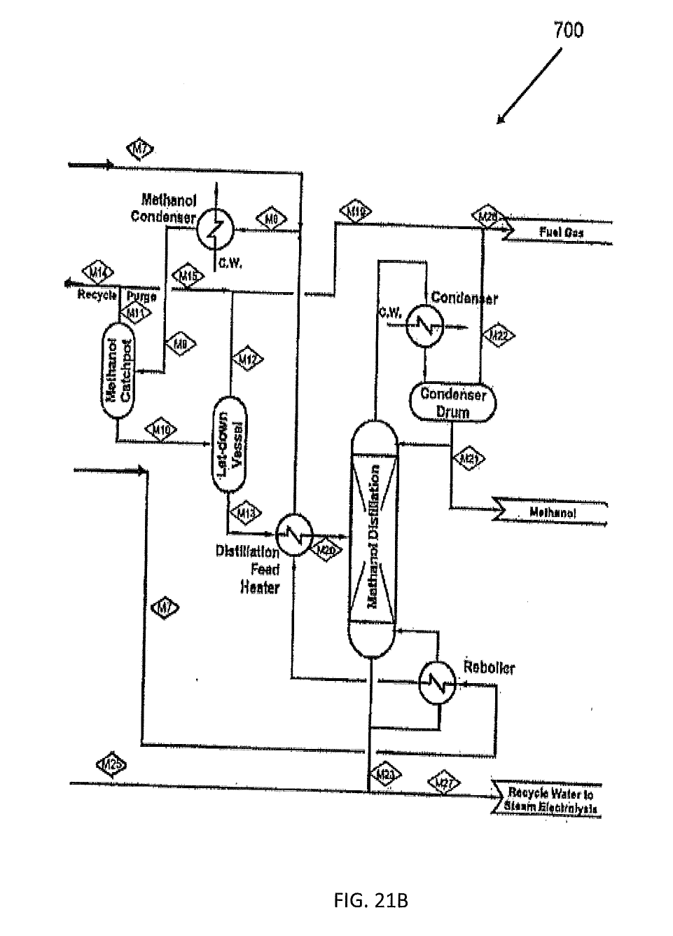

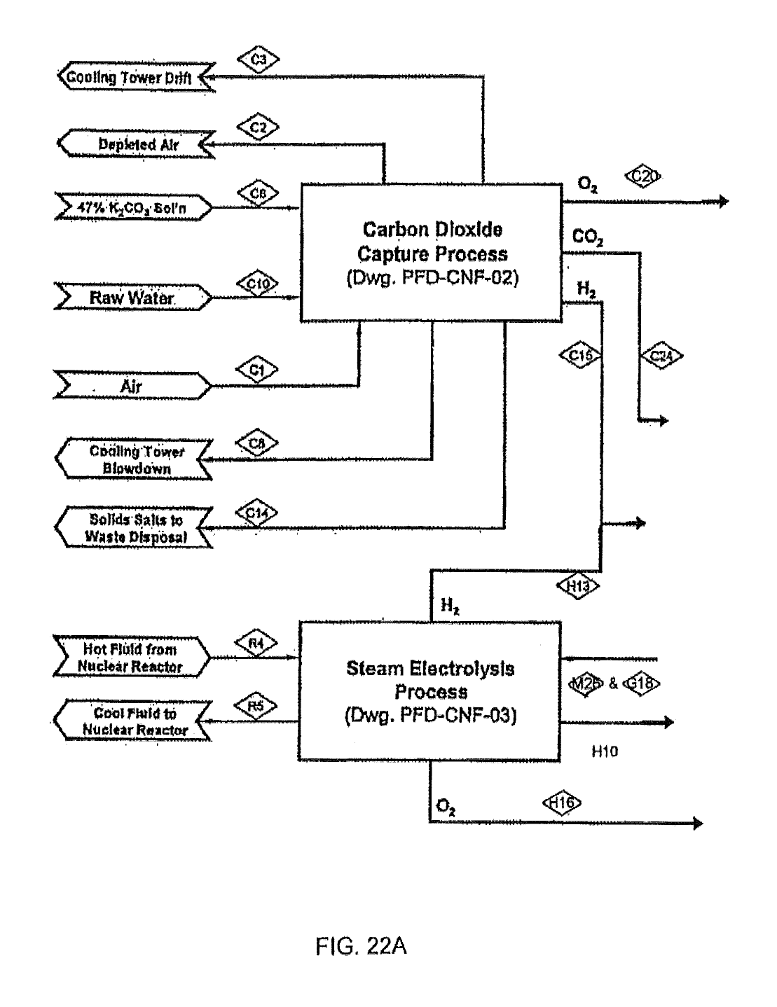

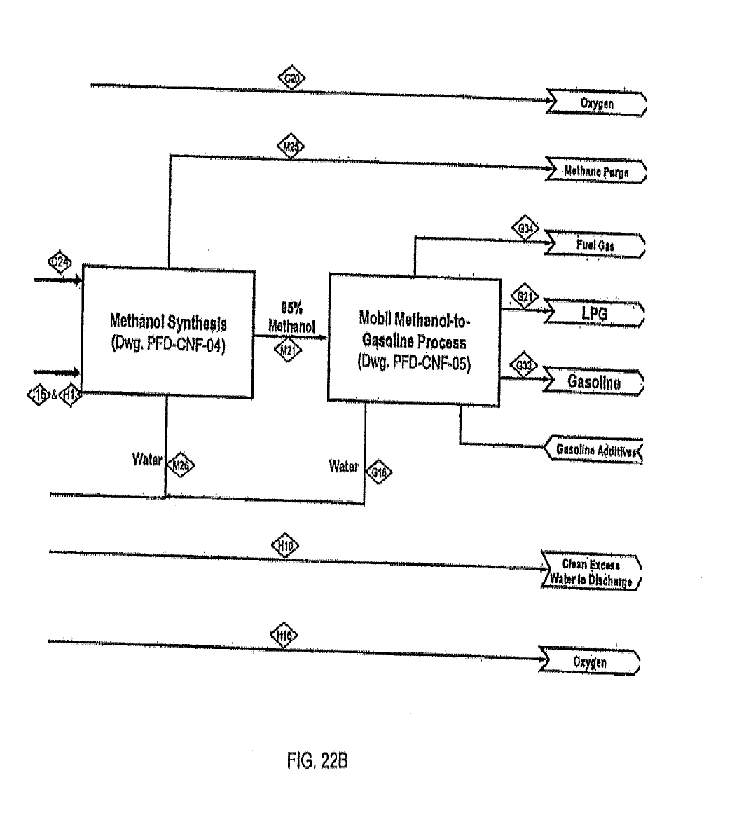

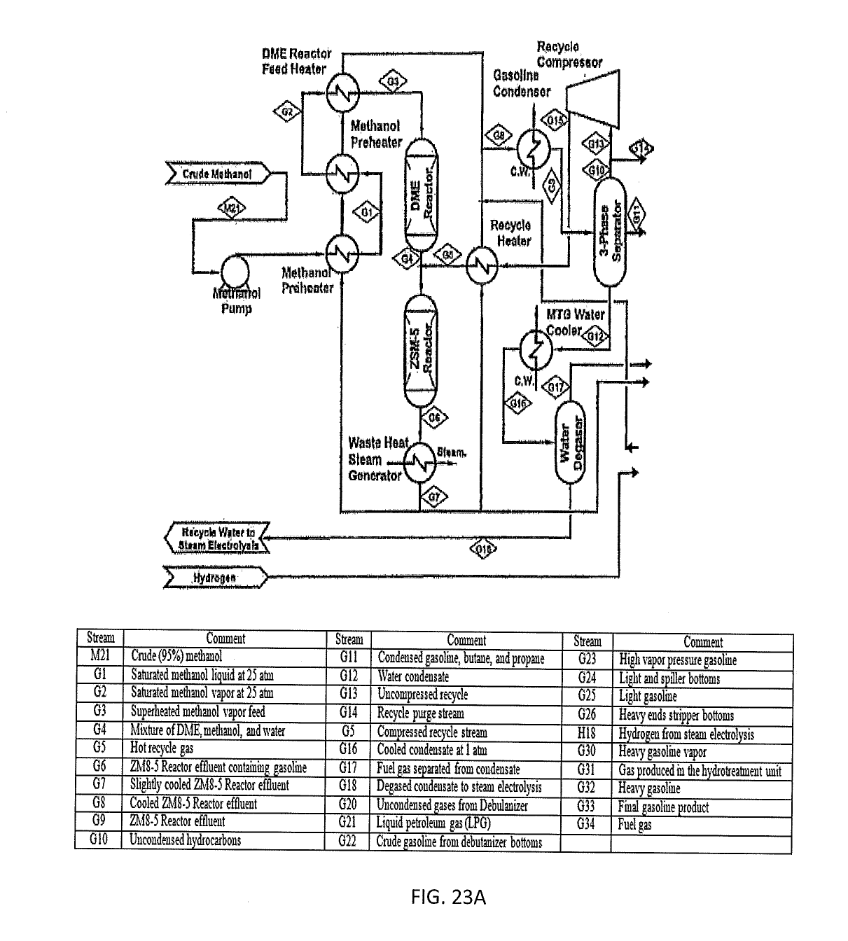

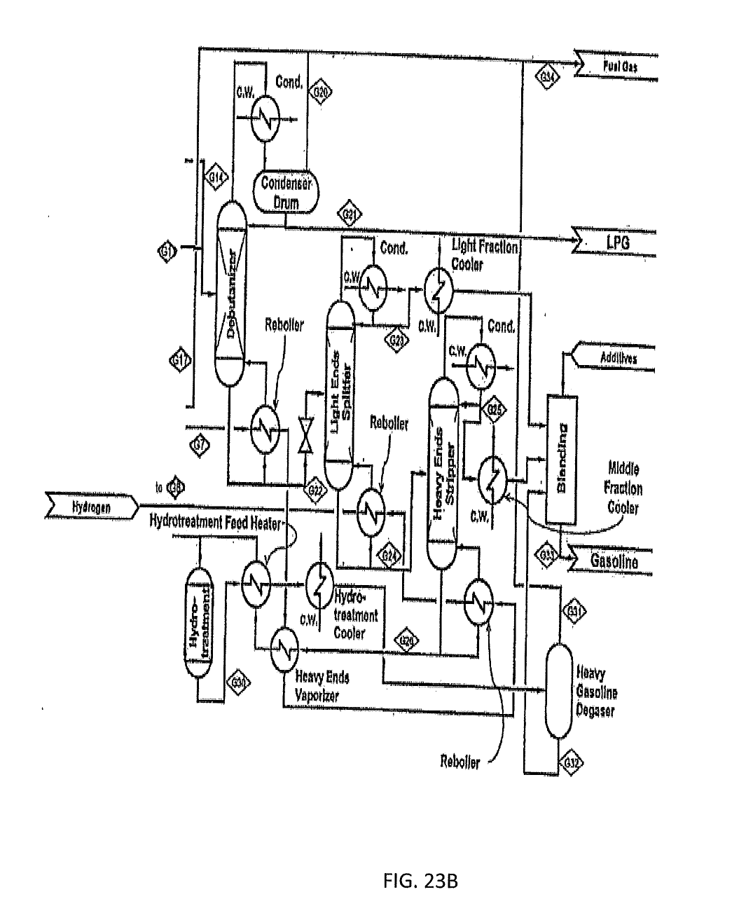

In one embodiment, illustrated by FIG. 21, carbon dioxide gas extracted by extracting step 300 and hydrogen gas produced by extracting step 300 are processed through a low pressure methanol compressor and then transferred to a high pressure methanol compressor. Hydrogen gas from the steam electrolysis process of hydrogen producing step 600 is also added to the high pressure methanol compressor and the synthesis gas is produced. That synthesis gas is then converted into methanol. Referring to FIG. 22 as an illustrative example of one embodiment of the method of the present invention, the methanol produced from the methanol synthesis process (detailed in FIG. 21) is then processed by the Mobil® Methanol-to-Gasoline Process to produce a synthetic gasoline. In another embodiment, referring to FIG. 23, methanol produced from the methanol synthesis (detailed in FIG. 21) is then processed by a fixed-bed methanol-to-gasoline process to produce synthetic gasoline.

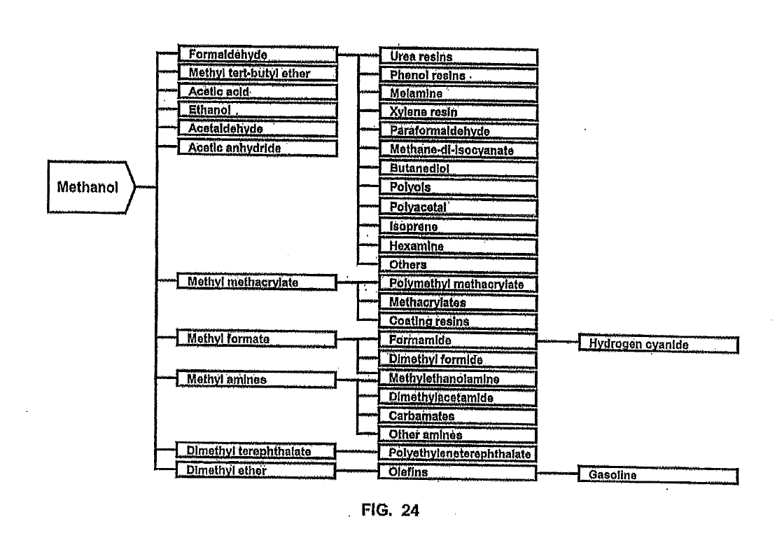

FIG. 24 illustrates the possible chemicals that can be produced from methanol including, but not limited to, formaldehyde, methyl tert-butyl ether, acetic acid, ethanol, acetaldehyde, acetic anhydride, methyl methacrylate, methyl formate, methyl amines, dimethyl terephthalate, dimethyl ether, mixtures thereof, and derivatives thereof. Formaldehyde may be used to produce urea resins, phenol resins, melamine, xylene resin, paraformaldehyde, methane-di-isocyanate, butanediol, polyols, polyacetal, isoprene, hexamine, and other substances. Methyl methacrylate may be used to further produce polymethyl methacrylate, methacrylates, coating resins, and other substances. Methyl formate may be used to further produce formamide which in turn may be used to produce hydrogen cyanide, dimethyl formide, methylethanolamine, and other substances. Methyl amines may be used to further produce methylethanolamine, dimethylacetamide, carbamates, and other amines. Dimethyl terephthalate may further be used to produce polyethyleneterephthalate, and other substances. Dimethyl ether may further be used to produce olefins, plastics, gasoline, and other substances.

In an illustrative example of how the method of the present invention may be used in a synthetic gasoline plant, the following sample process is given. It will be appreciated by one skilled in the art that this is merely one of countless possibilities for application of the method of the present invention. Carbon dioxide gas is extracted using a cooling tower absorber and electrolytic stripping. Hydrogen is also produced as a byproduct. Steam electrolysis is used to generate additional hydrogen gas. Hydrogen gas and carbon dioxide gas are converted into methanol. The Mobil® Methanol-to-Gasoline process is used to convert the methanol to gasoline. Nuclear power is used as the power source. The process is powered by a pressurized water reactor. The absorption solution is potassium carbonate and is also used for cooling the reactor. The reactor generates DC power tailored for the electrolytic stripping process and steam electrolysis. The reactor also provides steam to power the process compressor and provide the process heat. Special features of this example include that waste heat from the steam electrolysis process is used to superheat the feed to the steam electrolysis cell, the steam electrolysis cell is operated at a pressure corresponding to the suction of the high-pressure casing of the methanol synthesis-gas compressor, and waste heat from the methanol process is used to generate steam for steam electrolysis. This example process has the capacity to produce about 4430 bbl/day of gasoline from chemical plant and 1.64 GW thermal energy from the nuclear reactor. The energy used by this example process is about 221 MJ/gal of electrical energy per gallon of gasoline, about 89 MJ/gal of steam energy per gallon of gasoline, and about 673 MJ/gal of total thermal energy per gallon of gasoline. The energy efficiency of this example process is about 20% calculated by taking 100%×fuel value of gasoline÷thermal energy generated. The second law efficiency of this example process is about 43% calculated by taking 100%×minimum thermal energy required to produce gasoline÷actual thermal energy generated.

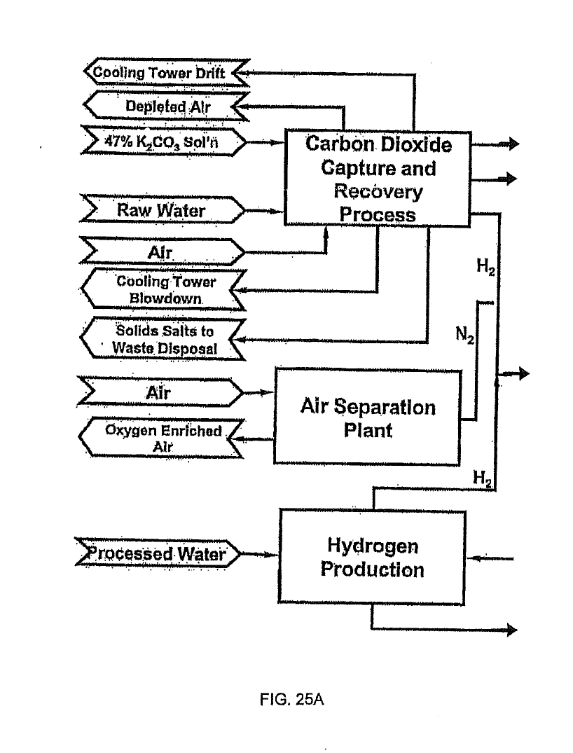

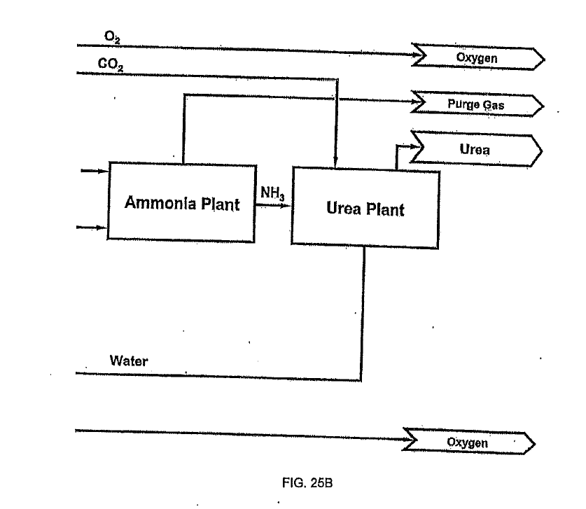

In an alternative embodiment, illustrated by FIG. 25, a method of producing urea from atmospheric carbon dioxide is provided and includes, first, an extracting step in which carbon dioxide gas is extracted from the atmosphere using the extracting step previously disclosed hereinabove. Then, in a hydrogen producing step, hydrogen gas is produced using water electrolysis, steam electrolysis, thermochemical process, or an iodate-sulfate process. Third, in a nitrogen producing step, air is processed through an air separation plant to produce nitrogen gas. Fourth, in a combining step, nitrogen gas from the nitrogen producing step, hydrogen gas from the extracting step, and hydrogen gas from the hydrogen producing step are combined together and then converted into ammonia using the Haber process which is incorporated herein by reference. Finally, in a converting step, the ammonia is combined with the carbon dioxide gas from the extracting step to produce urea.

It will be appreciated that additional steps and equipment may be utilized by the method of the present invention even though not discussed in detail herein. Additional equipment and processes may include, but are not limited to, a water treatment plant, process and power cycle coolers, filters, an anolyte recycle tank, an absorber feed cooler, a stripper-absorber interchanger, a reboiler, a condenser drum, a condenser, solution polishing, an electrolytic cell interchanger, an electrolytic cell feed heater, a catholye recycle tank, a fan within the nuclear cooling tower, recycling tanks for various solutions, a feedwater preheater, a superheater, a recycle compressor, a water catchpot, interstage coolers, a low pressure methanol compressor, a high pressure methanol compressor, a methanol recycle compressor, a methanol catchpot, a let-down vessel, methanol distillation, a distillation feed heater, a steam electrolysis boiler, a methanol condenser, methanol synthesis, a DME reactor feed heater, a methanol pump, a methanol preheater, a ZSM-5 reactor, a DME reactor, a gasoline condenser, a 3-phase separator, a water degaser, a MTG water cooler, a debutanizer, a hydrotreatment feed heater, hydrotreatment, a heavy ends vaporizer, a hydrotreatment cooler, a light ends splitter, a light fraction cooler, a heavy ends stripper, blending, and a heavy gasoline degaser.

Having described the invention in detail, those skilled in the art will appreciate that modifications of the invention may be made without departing from the spirit and scope thereof. Therefore, it is not intended that the scope of the invention be limited to the specific embodiments and examples described. Rather, it is intended that the appended claims and their equivalents determine the scope of the invention.

What is claimed is:

1. A method for producing a chemical product comprising the steps of:
extracting carbon dioxide gas from the atmosphere;
producing hydrogen gas;
combining said carbon dioxide gas and said hydrogen gas to produce a synthesis gas; and
converting said synthesis gas to said product.

2. The method of claim 1 wherein said method is powered by a power source selected from the group consisting of nuclear power, hydroelectric power, geothermal power, wind power, photovoltaic solar power, thermal solar power, and combinations thereof.

3. The method of claim 1 wherein said product is selected from the group consisting of fuel, diesel fuel, jet fuel, gasoline, petrochemicals, plastics, butane, methanol, ethylene, propylene, aromatic compounds, petrochemical derivatives, derivatives thereof, and mixtures thereof.

4. The method of claim 1 wherein said product further undergoes a process to convert said product to a fuel, wherein said process is selected from the group consisting of Synthesis Gas-to-Methanol, Methanol-to-Gasoline, Methanol-to-Olefins, Fischer Tropsch wax conversion, Fischer-Tropsch, and Fischer-Tropsch oil refining.

5. The method of claim 1 wherein said extracting step further comprises the steps of:
absorbing said carbon dioxide gas using an absorbent solution;
stripping said carbon dioxide gas from said absorbent solution, wherein said stripping step produces a mixture; and
separating said carbon dioxide gas from said mixture.

6. The method of claim 5 wherein said absorbent solution is selected from the group consisting of lithium carbonate, potassium carbonate, cesium carbonate, rubidium carbonate, francium carbonate, ammonium carbonate, beryllium carbonate, magnesium carbonate, calcium carbonate, strontium carbonate, barium carbonate, potassium carbonate and potassium hydroxide mixture, sodium carbonate and sodium hydroxide mixture, and mixtures thereof.

7. The method of claim 5 wherein said absorbing step uses a gas-contacting device to capture said carbon dioxide gas, wherein air is circulated in said gas-contacting device, and wherein said air comes in contact with said absorbent solution, and said carbon dioxide gas is absorbed by said absorbent solution to produce a solvent containing said absorbed carbon dioxide gas.

8. The method of claim 7 wherein said gas-contacting device is selected from the group consisting of nuclear cooling towers, natural draft cooling towers, assisted draft cooling towers, forced-draft cooling towers, absorption columns, absorption columns with trays, absorption columns with random packing, absorption columns with structure packing, hollow-fiber absorbers, cooling ponds, spray ponds, natural alkaline lakes, and combinations thereof.

9. The method of claim 5 wherein said stripping step uses an electrolytic cell to separate carbon dioxide gas from said absorbent solution, wherein separation produces said mixture, and wherein said mixture comprises carbon dioxide gas and oxygen gas.

10. The method of claim 9 wherein said electrolytic cell is selected from the group consisting of a hydroxide cell, a bicarbonate cell, a three compartment cell, and a mercury cell.

11. The method of claim 9 wherein said electrolytic cell comprises:
an anode compartment having an anode;
a cathode compartment having a cathode;
a membrane, wherein said membrane separates said anode compartment and said cathode compartment.

12. The method of claim 11 wherein said membrane is selected from the group consisting of a diaphragm, an ion-exchange membrane, a cation-exchange membrane, and an anion-exchange membrane.

13. The method of claim 11 wherein said electrolytic cell is a hydroxide cell comprising:
a first feed to said anode compartment wherein said solvent is fed through said first feed;
a second feed to said cathode compartment wherein a hydroxide solution is circulated therethrough;
a first product produced at said anode wherein said first product is said gas mixture;
a second product produced at said anode wherein said second product is a solution having an increased bicarbonate concentration;
a third product produced at said cathode wherein said third product is hydrogen gas; and
a fourth product produced at said cathode wherein said fourth product is a hydroxide solution.

14. The method of claim 11 wherein said electrolytic cell is a bicarbonate cell comprising:
a first feed to said cathode compartment wherein said solvent is fed through said first feed;
a second feed to said anode compartment wherein a carbonate solution is circulated therethrough;
a first product produced at said anode wherein said first product is said gas mixture;
a second product produced at said anode wherein said second product is a solution having an increased bicarbonate concentration;
a third product produced at said cathode wherein said third product is hydrogen gas; and
a fourth product produced at said cathode wherein said fourth product is a carbonate solution.

15. The method of claim 5 wherein said separating step separates said carbon dioxide from said mixture using a device selected from the group consisting of a carbonate scrubber, an amine scrubber, a pressure swing absorber, and a membrane separator.

16. The method of claim 1 wherein said producing hydrogen gas step produces hydrogen using a process selected from the group consisting of steam electrolysis, thermochemical process, iodate-sulfate process, and water electrolysis.

17. A method for producing a chemical product comprising the steps of:
extracting carbon dioxide gas from the atmosphere, wherein said extracting step further includes the steps of:
absorbing said carbon dioxide gas using an absorbent solution,
stripping said carbon dioxide gas from said absorbent solution and producing a gas mixture, and
separating said carbon dioxide gas from said gas mixture;
producing hydrogen gas;
combining said carbon dioxide gas and said hydrogen gas to produce a synthesis gas; and
converting said synthesis gas to said product.

18. A method for producing synthetic gasoline comprising the steps of:
absorbing carbon dioxide from the atmosphere using a nuclear cooling tower containing a packing soaked with an absorbent alkaline solution, wherein air is circulated through said nuclear cooling tower absorbing said carbon dioxide in said solution;
collecting said solution from a pond below said cooling tower;
stripping said carbon dioxide from said solution using an electrolytic cell, wherein said electrolytic cell produces a gas mixture containing carbon dioxide gas;
separating said carbon dioxide from said gas mixture;
producing hydrogen gas using a steam electrolysis process;
combining said carbon dioxide gas with said hydrogen gas to produce a synthesis gas;
converting said synthesis gas into methanol; and
converting said methanol into synthetic gasoline.

19. A method of claim 18 wherein said methanol is converted into synthetic gasoline using a process selected from the group consisting of Synthesis Gas-to-Methanol, Methanol-to-Gasoline, Methanol-to-Olefins, Fischer Tropsch wax conversion, Fischer-Tropsch, and Fischer-Tropsch oil refining.

20. A method of producing urea from atmospheric carbon dioxide comprising the steps of:
extracting carbon dioxide gas from the atmosphere;
producing hydrogen gas;
producing nitrogen gas;
converting nitrogen gas and hydrogen gas to ammonia;
combining ammonia with said carbon dioxide to produce urea.

21. A device for producing a chemical product comprising:
a nuclear reactor plant having a cooling tower; and
a chemical plant having an electrolytic cell, a carbon dioxide separator, an electrolysis device, and a converter,
wherein said nuclear reactor plant provides energy to said chemical plant.

22. The device of claim 21 wherein said cooling tower comprises a tower, a packing material, and a collection pond, wherein said packing material includes an absorbent solution, and wherein air is circulated through said cooling tower such that said air comes in contact with said absorbent solution thereby producing a solvent having at least carbon dioxide gas.

23. The device of claim 22 wherein said absorbent solution is selected from the group consisting of lithium carbonate, potassium carbonate, cesium carbonate, rubidium carbonate, francium carbonate, ammonium carbonate, beryllium carbonate, magnesium carbonate, calcium carbonate, strontium carbonate, barium carbonate, potassium carbonate and potassium hydroxide mixture, sodium carbonate and sodium hydroxide mixture, and mixtures thereof.

24. The device of claim 22 wherein said electrolytic cell removes carbon dioxide from said solvent and produces a gas mixture comprising carbon dioxide, and wherein said electrolytic cell is selected from the group consisting of a hydroxide cell, a bicarbonate cell, a three compartment cell, and a mercury cell.

25. The device of claim 21 wherein said product is selected from the group consisting of fuel, diesel fuel, jet fuel, gasoline, petrochemicals, plastics, butane, methanol, urea, ethylene, propylene, aromatic compounds, petrochemical derivatives, derivatives thereof, and mixtures thereof.
